(19) United States
(12) Patent Application Publication
(10) Pub. No.: US 2020/0121697 A1
(43) Pub. Date: Apr. 23, 2020

(54) METHODS FOR DIAGNOSING AND TREATING CACHEXIA

(71) Applicant: Aurin Biotech Inc., Vancouver (CA)

(72) Inventors: Patrick L. MCGEER, Vancouver (CA); Moonhee LEE, Vancouver (CA); Edith G. MCGEER, Vancouver (CA); Krista KENNEDY, North Vancouver (CA)

(21) Appl. No.: 16/605,785

(22) PCT Filed: May 30, 2018
(86) PCT No.: PCT/CA2018/050639

§ 371 Date: Oct. 16, 2019

Related U.S. Application Data
(60) Provisional application No. 62/513,277, filed on May 31, 2017.

(51) Int. Cl. A61K 31/603; G01N 33/68; A61K 31/616

(52) U.S. Cl. CPC A61K 31/603; G01N 33/6863; G01N 2333/525; A61K 31/616; G01N 33/6893

(57) ABSTRACT

A method of treating cachexia is provided. The method includes administering to a human in need thereof an effective amount of a complement inhibitor. The complement inhibitor may be a dimer of acetyl salicylic acid, including 5,5′-methylenebis(2-hydroxybenzoic acid), 4,4′-diacetoxy-1,1 biphenyl-3,3′ dicarboxylic acid or a pharmaceutically acceptable salt thereof. The complement inhibitor may be aurin tricarboxylic acid, aurin quadracarboxylic acid, aurin pentacarboxylic acid, aurin hexacarboxylic acid, combinations thereof, and pharmaceutically acceptable salts thereof. The complement inhibitor may be an ester prodrug of the foregoing compounds.

### TECHNICAL FIELD

This invention relates to diagnosing and treating cachexia, including cancer cachexia.

### BACKGROUND

Cachexia is a complex pathological syndrome with symptoms including weight loss, muscle atrophy, appetite loss and fatigue. Tumor necrosis factor alpha (TNFα), also termed cachexin, is a cytokine associated with cachexia. Cachexia affects the majority of terminal cancer patients, and increases patient morbidity and mortality. Cachexia is often the immediate cause of death itself in cancer patients. Cachexia is also seen in non-cancer contexts, including in HIV/AIDS, end-stage renal failure, chronic obstructive pulmonary disease (COPD), and geriatric cachexia.

In the 1970s Carswell et al. (1975) identified a protein which appeared in mouse serum following subcutaneous injection of a malignant sarcoma named BALB/c ascites sarcoma Meth A. When such serum was injected into mice with tumor transplants, it caused destruction of the tumors. They named the material in the serum TNFα. Host cells, particularly macrophages, were thought to be responsible for its generation. Beutler et al. (1985) subsequently showed that TNFα was identical with the macrophage-secreted factor cachexin.

The discovery of TNFα as an endogenously produced factor which could attack cancer cells set off a plethora of studies involving different types of cancers and different paradigms of experimentation. For example, this led to a clinical trial by Selby et al. (1987) of TNFα in advanced cancer cases with disastrous results. These, and other apparently inexplicable results, led to contradictory conclusions as to the properties of TNFα and confusion regarding the reasons for such contradictions. The situation has been comprehensively reviewed by Balkwill (2009). He noted that “unlike their normal counterparts, many malignant cells constitutively produce small amounts of TNF”, a notion shown by the inventors herein to be incorrect.

There is a general desire to provide improved methods for diagnosing and treating cachexia.

### SUMMARY

Aspects of the invention relate to treating cachexia through administration of complement inhibitors to reverse elevated TNFα production associated with cachexia. In particular embodiments, complement inhibitors dimers of acetyl salicylic acid (such as 4,4′-diacetoxy-1,1 biphenyl-3,3′ dicarboxylic acid (DAS)) and aurin carboxylic acids (such as aurin polycarboxylic acid complex (APAC)). Oral administration of complement inhibitors have been shown to restore TNFα production to control levels, terminating the cachexic state.

Types of cachexia treated include terminal stage cancer, as well as non-cancer conditions such as HIV/AIDS, end-stage renal failure, chronic obstructive pulmonary disease (COPD), and geriatric cachexia.

Aspects of the invention also relate to diagnosing and monitoring cachexia through determining salivary levels of TNFα. The inventors have determined that TNFα is produced at a low level by all organs of the body, presumably to establish and maintain the capillaries that provide their blood supply. The inventors show that TNFα is secreted in saliva, which is indicative of mandibular gland production, and can act as an indicator of production in brain or other organs. Salivary TNFα production has been determined to be constant in healthy individuals and elevated in patients with cachexia. Types of cachexia that can be diagnosed and monitored include terminal stage cancer, as well as non-cancer conditions such as HIV/AIDS, end-stage renal failure, chronic obstructive pulmonary disease (COPD), and geriatric cachexia.

An aspect of the invention provides a method of treating cachexia comprising administering to a human in need thereof an effective amount of a complement inhibitor. The complement inhibitor may be a dimer of acetyl salicylic acid or a pharmaceutically acceptable salt thereof. The dimer of acetyl salicylic acid may be 4,4′-diacetoxy-1,1 biphenyl-3,3′ dicarboxylic acid (DAS), 5,5′-methylenebis(2-hydroxybenzoic acid), or pharmaceutically acceptable salts thereof. The complement inhibitor may also be selected from the group consisting of aurin tricarboxylic acid
(5,5′-((3-carboxyl-4-oxocyclohexa-2,5-diene)methylene)bis(2-hydroxybenzoic acid)), aurin quadracarboxylic acid
((Z)-3-(3-carboxyl-4-hydroxybenzyl)-5-((3-carboxyl-4-hydroxyphenyl) (3-carboxyl-4-oxocyclohexa-2,5-dien-1-ylidene)methyl)-2-hydroxybenzoic acid), aurin pentacarboxylic acid
((Z)-5-(3-carboxyl-4-hydroxybenzyl)-3-((3-carboxyl-5-((3-carboxyl-4-hydroxyphenyl)(3-carboxyl-4-oxocyclohexa-2,5-dien-1-ylidene)methyl)-2-hydroxybenzyl)-2-hydroxybenzoic acid), aurin hexacarboxylic acid
((Z)-5, 5′-((3-carboxyl-5-(3-carboxyl-5-((3-carboxyl-4-hydroxyphenyl)(3-carboxyl-4-oxocyclohexa-2,5-dien-1-ylidene)methyl)-2-hydroxybenzyl)-4-hydroxyphenyl)(hydroxyl) methylene)bis(2-hydroxybenzoic acid)), or any combination of aurin tri-, quadra-, penta- and hexacarboxylic acids, and pharmaceutically acceptable salts thereof. The complement inhibitor may comprise aurin tri-, quadra-, penta- and hexacarboxylic acids, or pharmaceutically acceptable salts thereof. The complement inhibitor may alternatively be an inhibitor of formation of the membrane attack complex C5b-9. The complement inhibitor may be an ester prodrug of a dimer of acetyl salicylic acid, or aurin tri-, quadra-, penta- or hexacarboxylic acid, or a pharmaceutically acceptable salt thereof. The complement inhibitor may be administered orally. The cachexia may be cancer cachexia. The cachexia may be a non-cancer cachexia associated with a disorder selected from the group consisting of HIV/AIDS, end-stage renal failure, chronic obstructive pulmonary disease (COPD), and geriatric cachexia.

An aspect of the invention provides a method for diagnosing cachexia in a subject in need thereof, the method comprising: obtaining a saliva sample from the subject; stabilizing the saliva sample; determining a level of TNFα present in the pretreated saliva sample; comparing the determined value of the TNFα with that of a control value of TNFα derived from a saliva sample of an unaffected control group sample; and displaying the comparison of the determined value and the control value, wherein the determined value being greater than the control value is indicative of cachexia in the subject. The determined value being at least 1.5 times greater than the control value may be indicative of cachexia in the subject. The cachexia may be cancer cachexia. The cachexia may be a non-cancer cachexia associated with a disorder selected from the group consisting of HIV/AIDS, end-stage renal failure, chronic obstructive pulmonary disease (COPD), and geriatric cachexia. The control value of TNFα may range from 150 pg/ml to 175 pg/ml.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a Western blot showing the effect of APAC on TNFα stimulation of C3 expression in the human breast cancer cell line BT 474. Lanes 1-3 are 3 independent experiments using 3 untreated batches of BT474 cells. Lanes 3-6 are the same batches after treatment for 24 hours with 1 ng/ml TNFα. C3 production is substantially upregulated by the TNFα. Lanes 7-9 are the same batches treated in the same way but with the addition of 30 μM APAC. The upregulated C3 production is blocked indicating that APAC is an inhibitor of TNFα stimulation of C3 production in the BT 474 breast cancer cell line.

FIG. 2 is a Western blot showing the effect of APAC on TNFα stimulation of C3 and C9 expression in the human pancreatic cell line BxPC3. Lanes 1-3 are 3 independent experiments using 3 untreated batches of BxPC3 cells. Lanes 3-6 are the same batches after treatment for 24 hours with 1 ng/m1 TNFα. C3 and C9 production is substantially upregulated by the TNFα. Lanes 7-9 are the same batches treated in the same way but with the addition of 30 μM APAC . The upregulated C3 and C9 production is blocked indicating that APAC is an inhibitor of TNFα stimulated production of C3 and C9 in the human pancreatic cancer cell line.

FIG. 3 is a Western blot showing the effect of APAC on TNFα stimulation of C3 and C9 expression in the human colon cancer cell line LS180. Lanes 1-3 are 3 independent experiments using 3 untreated batches of LS180 cells. Lanes 3-6 are the same batches after treatment for 24 hours with 1 ng/m1 TNFα. C3 and C9 production is substantially upregulated by the TNFα. Lanes 7-9 are the same batches treated in the same way but with the addition of 30 μM APAC . The upregulated C3 and C9 production is blocked indicating that APAC is an inhibitor of TNFα stimulated production of C3 and C9 in the human LS180 colon cancer cell line.

FIG. 4 is a Western blot showing the effect of APAC on TNFα stimulation of C3 and C9 expression in human microglia. Lanes 1-3 are 3 independent experiments using 3 untreated batches of human microglia. Lanes 3-6 are the same batches after treatment for 24 hours with 1 ng/ml TNFα. C3 and C9 production are upregulated by the TNFα. Lanes 7-9 are the same batches treated in the same way but with the addition of 30 μM APAC. The upregulated C3 and C9 production is blocked indicating that APAC is an inhibitor of TNFα stimulated production of C3 and C9 in the human microglia.

FIG. 5 is a Western blot showing the effect of APAC on TNFα stimulation of C3 and C9 expression in human astrocytes. Lanes 1-3 are 3 independent experiments using 3 untreated batches of human microglia. Lanes 3-6 are the same batches after treatment for 24 hours with 1 ng/ml TNFα. C3 and C9 production are not upregulated by the TNFα. Lanes 7-9 are the same batches treated in the same way but with the addition of 30 pM APAC. Again there is no effect. These data indicate that TNFα does not stimulate production of C3 and C9 in human astrocytes.

FIG. 6 is a graph plotting an ELISA kit readout of TNFα in the concentration range from zero to 1000 pg/ml. These data demonstrate a linear relationship between absorbance values at 450 nm and TNFα in pg/ml in the range of zero to 1,000 pg/ml.

FIG. 7 is a photograph showing immunohistochemical detection of TNFα in the cerebral cortex of a patient who died from pancreatic cancer. TNFα was immunohistochemically detected using the MyBiosource rabbit polyclonal antibody (MBS7002356) as the primary antibody at 1:1000 dilution). Notice the prominent heavy immunostaining of capillaries.

FIG. 8 is a photograph showing immunohistochemical detection of TNFα in the cerebral cortex of a patient who died from a sudden heart attack. TNFα was immunohistochemically detected using the MyBiosource rabbit polyclonal antibody (MBS7002356) as the primary antibody at 1:1000 dilution). There is only faint background staining.

FIG. 9 is a graph plotting binding of APAC to a TNFα peptide as a function of the peptide concentration. Binding of APAC is linear in the range of 0-5 nmol/ml of APAC and 0-175 nmol/ml of TNFα peptide.

FIG. 10 is a graph plotting binding of DAS to a TNFα peptide as a function of the peptide concentration. Binding of DAS is linear in the range of 0-5 nmol/ml of DAS and 0-175 nmol/ml of TNFα peptide.

FIG. 11 is a graph plotting salivary TNFα levels over time of a patient suffering from terminal leukemia. Treatment with APAC reversed elevated salivary TNFα.

FIG. 12 is a graph plotting salivary TNFα levels over time of a patient suffering from terminal breast cancer. Treatment with APAC reversed elevated salivary TNFα.

FIG. 13 is a graph plotting salivary TNFα levels over time of a patient suffering from terminal ovarian cancer. Treatment with APAC reversed elevated salivary TNFα.

FIG. 14 is a graph plotting salivary TNFα levels over time of a patient suffering from terminal colon cancer. Treatment with DAS reversed elevated salivary TNFα.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The term “aurin polycarboxylic acid complex” or “APAC” refers to a mixture of low molecular weight (i.e., less than 1 kDa) components obtained from synthesized or commercially-obtained “aurin tricarboxylic acid”. The low molecular weight components include aurin tri-, quadra-, penta- and hexacarboxylic acids. Methods for obtaining APAC, also known as the aurin tricarboxylic acid complex (ATAC), are described in US patent publication no. US2015/0065573 and Lee et al. (2012), incorporated by reference herein in their entirety.

The term “cancer” as used herein refers to the physiological condition in mammals that is typically characterized by unregulated cell growth. The term “cancer” includes cancer of any origin, including benign and malignant cancers, metastatic and non-metastatic cancers, and primary and secondary cancers. The term “cancer” includes reference to cancer cells. Examples of cancers include, but are not limited to, cancers of the bladder, blood, bone, brain/CNS, breast, cervix, colon, duodenum, esophagus, eye, gall bladder, heart, kidney, larynx, liver, lung, mouth, ovary, pancreas, pharynx, prostate, rectum, stomach, testis, uterus, as well as AIDS-related cancers, Hodgkin's disease, non-Hodgkin's lymphoma, multiple myeloma, melanoma, leukemia (including lymphocytic leukemia, hairy cell leukemia, and acute myelogenous leukemia), choriocarcinoma, rhabdomyosarcoma, and neuroblastoma.

The term an “effective amount” as used herein refers to the amount of the active agent sufficient to elicit a desired biological response (or, equivalently, to inhibit an undesired biological response). As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses.

The term “prodrug” as used herein refers an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound. There are numerous reasons why a prodrug strategy is used in drug design, the most common of which are to overcome problems associated with the compound, such as solubility, absorption and distribution, site specificity, instability, prolonged release, toxicity, poor patient acceptability, and formulation. A common prodrug form for drugs containing alcohol or carboxylic acid functional groups is an ester. Methods of making prodrugs are well known (e.g. Balant (1990); Bundgaard (1991); Silverman (1992)).

The term “treat”, “treating” and “treatment” as used herein refers to relief, reduction or alleviation of at least one symptom of a disease or condition in a subject. For example, treatment can be diminishment of one or several symptoms of a disease or condition or complete eradication of a disease or condition.

Cancers may be relatively benign until yet to be identified factors induce TNFα production leading to cachexia. One possible reason is that, as cancer cells proliferate, the newly formed cells need TNFα to stimulate their necessary blood supply. The inventors have determined, as demonstrated herein, that TNFα stimulation also induces cells to upregulate production of complement. Such upregulation might cause them to generate and release vascular endothelial growth factor (VEGF). VEGF is a requirement for new endothelial cell formation. Complement upregulation may also provide enhanced protection of cancer cells by expressing elevated levels of such self-protective proteins as factor H, protectin (CD 59), decay accelerating factor, and possibly others.

Aspects relate to methods of treating cachexia by administration to a human subject of an effective amount of a dimer of acetyl salicylic acid, aurin tricarboxylic acid
(5,5′-((3-carboxyl-4-oxocyclohexa-2,5-diene)methylene)bis(2-hydroxybenzoic acid), MW 422), aurin quadracarboxylic acid
((Z)-3-(3-carboxyl-4-hydroxybenzyl)-5-((3-carboxyl-4-hydroxyphenyl) (3-carboxyl-4-oxocyclohexa-2,5-dien-1-ylidene)methyl)-2-hydroxybenzoic acid), MW 573), aurin pentacarboxylic acid
((Z)-5-(3-carboxyl-4-hydroxybenzyl)-3-((3-carboxyl-5-((3-carboxyl-4-hydroxyphenyl)(3-carboxyl-4-oxocyclohexa-2,5-dien-1-ylidene)methyl)-2-hydroxybenzyl)-2-hydroxybenzoic acid), MW 722), and aurin hexacarboxylic acid
((Z)-5,5′-((3-carboxyl-5-(3-carboxyl-5-((3-carboxyl-4-hydroxyphenyl)(3-carboxyl-4-oxocyclohexa-2,5-dien-1-ylidene)methyl)-2-hydroxybenzyl)-4-hydroxyphenyl)(hydroxyl) methylene)bis(2-hydroxybenzoic acid), MW 857), aurin polycarboxylic acid complex (APAC) (a mixture of aurin tricarboxylic acid, aurin quadracarboxylic acid, aurin pentacarboxylic acid and aurin hexacarboxylic acid), prodrugs thereof, or salts thereof.

In some embodiments, the administration route of the active agent, prodrugs thereof, or salts thereof, alone or in a composition, in terms of effect may be local or systemic (enteral or parenteral), and in terms of location may for example be buccal, epicutaneous, epidural, intraarticular, intracardiac, intracavernous, intracerebral, intracerebroventricular, intradermal, intramuscular, intraosseous, intraperitoneal, intrathecal, intrauterine, intravaginal, intravenous, intravesical, intravitreal, nasal, oral, rectal, subcutaneous, sublingual, sublabial, transdermal, transmucosal, and the like.

In some embodiments, oral administration may be in the form of capsules, cachets, pills, tablets, lozenges, pastes, powders, granules, or as a solution or a suspension in an aqueous or non-aqueous liquid, or as an oil-in-water or water-in-oil liquid emulsion, or as an elixir or syrup, or as pastilles or as mouth washes and the like. In some embodiments, compositions in solid dosage forms for oral administration include capsules, tablets, pills, dragees, powders, granules and the like. The solid dosage forms may be scored or prepared with coatings and shells, such as enteric coatings and other coatings. They may also be formulated so as to provide slow or controlled release of the active agent. In some embodiments, compositions in liquid dosage forms for oral administration include emulsions, microemulsions, solutions, suspensions, syrups and elixirs. In some embodiments, topical or transdermal administration may be in the form of powders, sprays, ointments, pastes, creams, lotions, gels, solutions, controlled-release patches and inhalants. In some embodiments, parenteral administration (e.g. intravenous administration) may be in the form of solutions in physiologically compatible buffers. In some embodiments oral administration may be in the form of a prodrug of the active agent.

Various cell types were exposed to TNFα stimulation in vitro. The cell types could be various cancer cell lines or non-malignant cells, such as microglia and astrocytes.

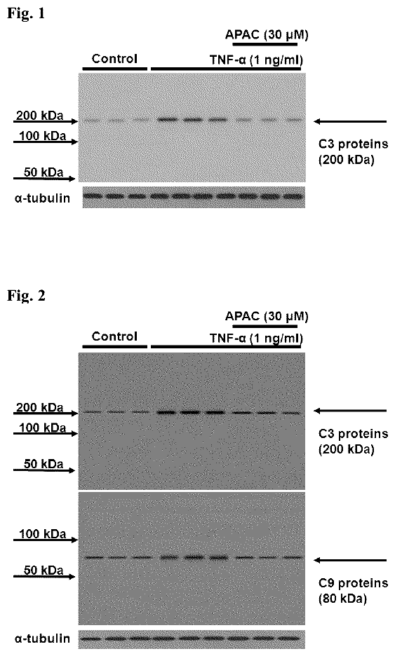

FIG. 1 is a Western blot illustrating the effect of TNFα on C3 expression in the human breast cancer cell line BT474. The cell line does not express C9. At 1 ng/ml, TNFα induced a substantial increase in C3 production which was blocked by APAC.

FIG. 2 is a Western blot illustrating the effect of TNFα on C3 and C9 expression in the human pancreatic cancer cell line BxPC-3. At 1 ng/ml, TNFα similarly induced a substantial increase in C3 and C9 production which was also blocked by APAC.

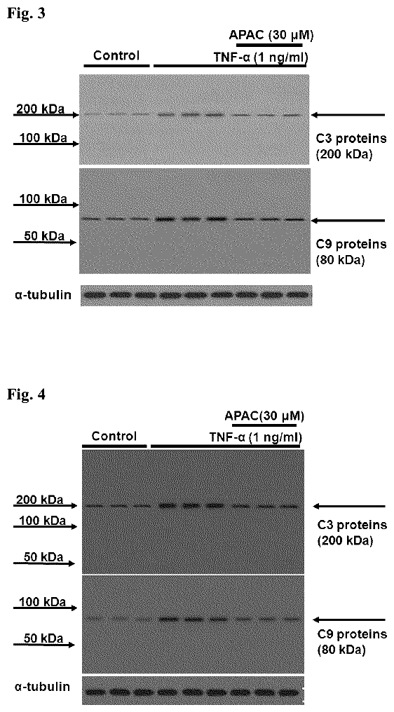

FIG. 3 is a Western blot of the colon cancer cell line LS180, showing a comparable effect on C3 and C9 for this malignant cell line which was also blocked by APAC.

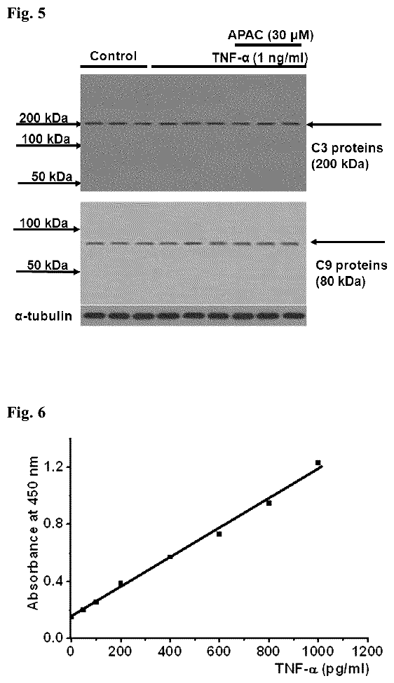

TNFα is also generated by normal tissue. FIG. 4 is a western blot showing the production of TNF by human microglia, while FIG. 5 shows a lack of production by human astrocytes.

Endogenous TNFα production can be measured by ELISA. The data reported herein used the Peprotech human TNFα kit (catalog 900-T25) following directions of the manufacturer. Alternative ELISA methods, not relying on commercial manufacturers, could be employed. FIG. 6 is a graph demonstrating a linear relationship between absorbance (at 450 nm) and added TNFα in the concentration range from zero to 1000 pg/ml. This standard curve was used to calculate TNFα production in various organs and in saliva as presented herein.

Table 1 shows that TNFα is produced by every tissue of the body that was measured. The values range from 21 pg/ml in the heart to 122 pg/ml in the kidney. Salivary TNFα levels can be taken as a surrogate for submandibular gland production of TNFα. Salivary TNFα levels in healthy volunteers shown in Table 2 demonstrate that this level is remarkably constant regardless of age, and ranges from 155-175 pg/ml. TNFα is substantially elevated in terminal cancer patients typically ranging from 405-462 pg/ml.

TABLE 1
TNF-α levels in human tissues

| Human tissue | TNF-α (pg/ml) |
|---|---|
| Lung | 162.39 |
| Heart | 21.44 |
| Kidney | 122.16 |
| Hippocampus | 31.52 |
| Sensory cortex | 33.38 |
| Liver | 72.48 |
| Spleen | 93.55 |
| Small intestine | 29.93 |
| Pancreas | 44.59 |

TABLE 2
Salivary TNF-α levels in normal humans

| Case No | Age | Gender | Diagnosis | TNF-α (pg/ml) |
|---|---|---|---|---|
| 1 | 54 | Female | Normal | 155.93 |
| 2 | 52 | Male | Normal | 168.11 |
| 3 | 53 | Male | Normal | 161.49 |
| 4 | 55 | Male | Normal | 166.48 |
| 5 | 43 | Female | Normal | 174.98 |
| 6 | 79 | Male | Normal | 162.69 |
| 7 | 54 | Male | Normal | 164.12 |
| 8 | 49 | Female | Normal | 156.17 |
| 9 | 71 | Male | Normal | 152.79 |
| 10 | 59 | Male | Normal | 157.92 |
| 11 | 77 | Male | Normal | 165.34 |
| 12 | 45 | Male | Normal | 156.34 |
| 13 | 47 | Female | Normal | 143.35 |
| 14 | 52 | Male | Normal | 166.51 |
| 15 | 74 | Female | Normal | 156.29 |
| 16 | 78 | Male | Normal | 146.93 |
| 17 | 12 | Male | Normal | 144.48 |
| 19 | 63 | Female | Normal | 159.62 |
| 20 | 59 | Male | Normal | 161.44 |
| 21 | 71 | Male | Normal | 162.56 |
| 22 | 59 | Male | Normal | 155.48 |
| 23 | 52 | Female | Normal | 161.61 |
| 24 | 58 | Male | Normal | 158.37 |
| 25 | 22 | Male | Normal | 161.26 |
| Total mean ± | | | | 159.18 ± 1.51 |
| SEM | | | | |

According to one embodiment, a method for diagnosing cachexia comprises: obtaining a saliva sample from a human patient; stabilizing the saliva sample; detecting a value of TNFα present in the pretreated saliva sample; comparing the detected value of the TNFα with a predetermined value; and displaying a comparison of the detected value and the predetermined value.

The stabilization step may involve known methods for stabilizing TNFα, such as those described by Lee et al. (2017), incorporated herein by reference.

The detection step is designed to treat the stabilized saliva sample with an TNFα antibody in such a way as to bind essentially all of the TNFα present in the sample. The detection step may involve an immunoassay, such as an ELISA test based on an antigen-antibody reaction using TNFα as the antigen to be measured. For example, the capture antibody may be a polyclonal antibody specific for TNFα. The bound TNFα is then detected by a second TNFα antibody which does not cross react with the first TNFα antibody. In some embodiments the second TNFα antibody may be a monoclonal antibody, such as a mouse monoclonal antibody. Biotin may then be bound to the monoclonal antibody or to a third IgG antibody to which biotin is bound. The biotin levels are detected by first treating with a streptavidin solution linked with horse radish peroxidase (HRP) followed by treatment with a tetramethylbenzidine solution to detect the HRP by reading the resulting color in a spectrophotometer. An ELISA standard concentration graph of target TNFα may be obtained, for example, by absorbance detection, fluorescence detection, luminescence detection, or electrochemical detection. FIG. 6 shows a standard concentration graph for TNFα obtained by absorbance at 450 nm. Alternatively, the immunoassay may be performed by a method capable of directly or indirectly detecting TNFα. Examples of such alternative methods include MS (Mass Spectrometry), MS/MS, and liquid chromatography.

The comparison step may involve comparing the detected level of TNFα obtained from the detection step against a predetermined level of TNFα. The predetermined level of TNFα may be obtained from concurrent or previous saliva samples from normal individuals without AD and without any genetic predisposition for AD.

The displaying step involves displaying values, diagrams, illustrations, and the like. This step is preferably a step that can assist in assessing the difference between the detected value and the predetermined value. For example, the difference may be displayed by the ratio of the detected value to the predetermined value. A diagnosis of cachexia may be made, for example, if this ratio exceeds 1.5, or 2.0, or 2.5 or 3.0 or 3.5 or 4.0.

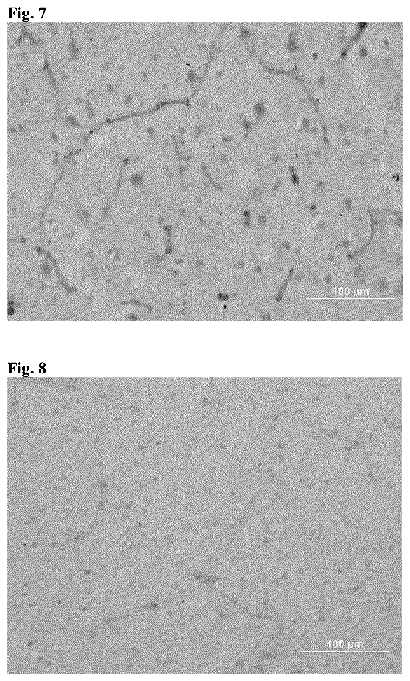

Evidence that TNFα stimulates vascular production in cancer patients comes from immunohistochemical studies of the brains of patients dying from terminal cancer. FIG. 7 shows TNFα immunohistochemical staining of the brain of a patient who died from pancreatic cancer using the MyBiosource rabbit polyclonal antibody (MBS7002356, 1:1000 dilution). There is heavy immunostaining of capillaries. FIG. 8 shows comparable staining of the brain of a patient who died from a sudden heart attack. There is only faint background staining. Detailed methodology is described in Schwab et al (2013), incorporated by reference herein.

A method for preparing dimers of acetyl salicylic acid is described in PCT publication no. WO 2015/070354 and Lee et al. (2015), incorporated by reference herein. An exemplary dimer of acetyl salicylic acid is 5,5′-methylenebis-2-acetoxybenzoic acid (DAS) and its isomers, 2-acetoxy-3-(4-acetoxy-3-carboxybenzyl)benzoic acid, 2-acetoxy-3-(3-acetoxy-4-carboxybenzyl)benzoic acid, 2-acetoxy-4-(4-acetoxy-3-carboxybenzyl)benzoic acid, 3,3′-methylenebis(2-acetoxybenzoic acid), 4,4′-methylenebis(2-acetoxybenzoic acid), are also within the scope of the invention.

To demonstrate that DAS and APAC inhibit TNFα activity by binding directly to TNFα, the inventors prepared an array of overlapping TNFα peptides to determine which TNFα sequences might block DAS and APAC binding to TNFα. A 12-mer peptide spot array was synthesized on a cellulose membrane using a Semi-automated AutoSpot robot (INTAVIS, USA) covering the 233 amino acid sequence of TNFα. A total of 112 peptides, each shifted by 2 amino acids were synthesized. The TNFα membrane was soaked in 50% methanol in 10 mM PBS (pH7.4) for 2 minutes, then washed twice with PBS. The membrane was then incubated with 1 mM of APAC or DAS in PBS (10 mM, pH7.4) for one hour at 37° C. After washing 3 times with PBS, positive images were captured with a SYPRO Ruby filter for APAC and an Alexa 680 filter for DAS by the Chemidoc MP imaging System (Bio-Rad, USA).

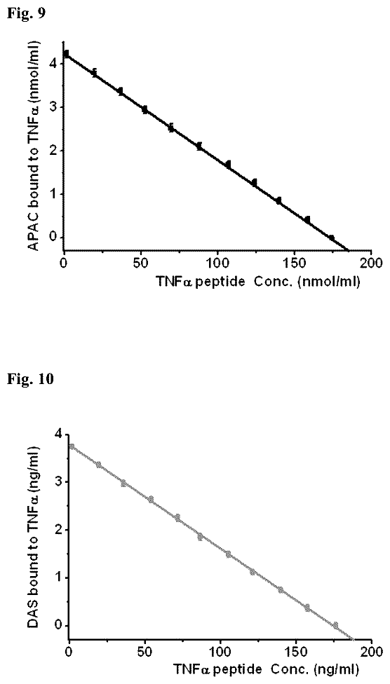

A strongly positive peptide sequence was chosen for synthesis of a potential inhibitor of TNFα binding. The sequence of this TNFα-A2 peptide was NH3-YLGGVFQLEKGDRLSA-COOH. It was synthesized using a standard peptide Synthesizer (Liberty Blue, CEM Inc, USA) with Wang Resin and Fmoc amino acids (GL Biochem Ltd, Shanghai, CN). After synthesis, the peptide was purified by HPLC on a C18 column (Quadarupole 6110 LC/MS, Agilent Technology, USA) with a flow phase of 0.5% trifluroacetic acid (TFA) in acetonitrile to 0.05% TFA in H₂O. The eluted peptide was detected by ultraviolet light (280 nm). The peptide inhibited binding of APAC to TNFα (FIG. 9) and of DAS to TNFα (FIG. 10). This finding does not rule out the possibility that APAC and DAS bind to other sequences in TNFα.

### EXAMPLES

The invention can be further understood by reference to the following examples, which are provided by way of illustration and are not meant to be limiting. The examples are of human cancer cases that had become cachexic. The patients had salivary TNFα levels ranging from 405 to 462 pg/ml. The TNFα in these cases was restored to normal levels by treatment with DAS (4,4′-diacetoxy-1,1 biphenyl-3,3′ dicarboxylic acid) or APAC for 10-21 days. These data indicate that individuals suffering from a cachexic state may be rescued by such treatment. They also support the fact that complement is required for TNFα production.

The inventors have determined that cancer patients who have deteriorated into a cachexic state have a sharp increase in their salivary levels of TNFα. This increase is rapidly reduced to normal levels by oral intake of complement inhibitors such as APAC and DAS.

These examples illustrate that cancer patients can be rescued from a cachexic state by a brief treatment with an inhibitor of the terminal complement pathway. The evidence suggests that rapidly proliferating cancer cells are heavily dependent upon increases in complement production to supply their vascular requirements.

### Example 1: Salivary TNFα Restoration to Normal with APAC Treatment

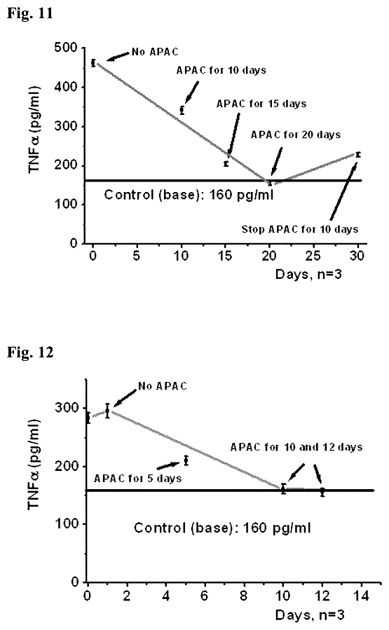

The patient was a 60 year old male diagnosed with terminal leukemia and cachexia. He was given a life expectancy of one month. Two weeks later the inventors obtained a saliva sample (Day 1) at the time of his biweekly blood transfusion. The TNFα level was found to be 454 pg/ml instead of the expected normal level of about 160 pg/ml. On Day 1 he began oral intake of 300 mg/3×/day of APAC. By Day 7 he no longer needed any blood transfusions, and his appetite and well-being improved. A second saliva sample was taken on Day 10 and the TNFα level had dropped to 349 pg/ml. A further sample was taken on Day 15 and the TNFα level was reduced to 205 pg/ml. A final sample was taken on Day 20 and at 155 pg/ml his TNFα level was down to normal levels and symptoms of cachexia subsided. On Day 20, following the advice of his oncologist, the patient discontinued APAC and began taking hydroxyurea (500 mg×2/day). Ten days later his TNFα saliva levels had risen to 228 pg/ml. Fifty days later he died. The data are shown in FIG. 11. The data indicate that continuing use of APAC may be necessary to maintain TNFα levels within the normal range and to avoid a recurrence of a cachexia.

### Example 2: Salivary TNFα Restoration to Normal with APAC Treatment

The patient was a 54 year old woman diagnosed with stage IV ductal breast carcinoma. She underwent a double mastectomy, chemotherapy and radiation therapy but the side effects from her treatments were so severe that she had bone pain, was unable to walk and was exhausted all the time. Her cancer continued to progress, she became cachexic, and her salivary TNFα was measured at 295 pg/ml. At this point she started taking APAC (3×300 mg/day). After 10 days on APAC (3×100 mg/day) her TNFα values had returned to normal at 160 pg/ml and symptoms of cachexia subsided (for example, her bone pain disappeared, and her strength restored to the point where she was able to travel overseas). The data are shown in FIG. 12.

### Example 3: Salivary TNFα Restoration to Normal after APAC

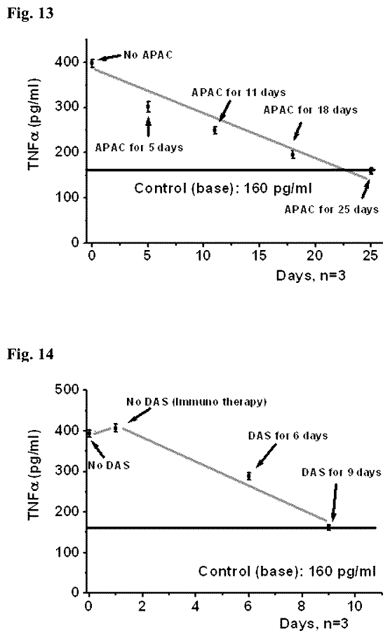

The patient was a 57 year old woman diagnosed with ovarian cancer and cachexia. She was recently treated with Re-Stimulated Autologous Tumor-Infiltrating Lymphocytes (TILS) followed by low-dose interleukin-2. The treatment was unsuccessful and it was anticipated that the subsequent scans would show metastatic tumor growth. She was on no treatment except for pain medications (17.5 mg methadone and 80 mg OxyNeo). That was also unsuccessful. Her TNFα saliva level was 394 pg/ml before she started taking APAC (100mg/3×/day). Her salivary TNFα level returned to normal sometime between 18 and 25 days and symptoms of cachexia subsided. The data are shown in FIG. 13.

### Example 4: Salivary TNFα Restoration to Normal after DAS

The patient was a 66 year old woman diagnosed with stage IV peritoneal carcinomatosis and cachexia. A saliva sample was obtained at this time and she was found to have an elevated TNFα level of 394 pg/ml (Day 0). A second sample was taken 3 days later and was found to have increased to 406 pg/ml. She was then started on DAS (3×100 mg/day). Five days into her treatment (Day 8) her TNFα level had dropped to 288 pg/ml. Nine days into her treatment (Day 12) her TNFα had returned to a normal level of 161 pg/ml and symptoms of cachexia subsided. The data are shown in FIG. 13.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

Balant, L. P., et al., 1990. Eur. J. Drug Metab. Pharmacokinet. 15:143-153
Balkwill, F. Tumor necrosis factor and cancer. Nat Rev Cancer 5, 361-71 (2009)
Beutler, B. et al. Identity of tumour necrosis factor and the macrophage-secreted factor cachectin. Nature 316, 552-554 (1985)
Bundgaard, H., et al., 1991. Drugs of the Future 16:443-458
Carswell, E. A. et al. An endotoxin-induced serum factor that causes necrosis of tumours. Proc. Natl Acad. Sci. USA 72, 3666-3670 (1975)
Lee M, Guo J P, Kennedy K, McGeer E G, McGeer P L. A method for diagnosing Alzheimer's disease based on salivary amyloid beta protein 42 levels. J Alz Dis. 55; 1175-1182 (2017)
Lee M, Wathier M, Love J A, McGeer E, McGeer P L. Inhibition of aberrant complement activation by a dimer of salicylic acid. Neurobiol Aging 36; 2748-2756 (2015)
Lee M, Guo J-P, Schwab C, McGeer E G, McGeer P L. Selective inhibition of the membrane attack complex of complement by low molecular weight components of the aurin tricarboxylic acid synthetic complex. Neurobiol Aging 33: 2237-2246 (2012)
Schwab C, Yu S, Wong W, McGeer E G, McGeer P L. GAD 65, GAD 67, and GABAT immunostaining in human brain and apparent GAD 65 loss in Alzheimer's disease. J Alz Dis 33: 1073-1088 (2013)
Selby P, Hobbs S, Viner C, Jackson E, Jones A, Newell D, Calvert A H, McElwain T, Fearon K, Humphreys J, et al. Br J Cancer.56(6):803-8 (1987)
Silverman, Richard B. The Organic Chemistry of Drug Design, Acad. Press, 1992

1. A method of treating cachexia comprising administering to a human in need thereof an effective amount of a complement inhibitor.

2. A method according to claim 1 wherein the complement inhibitor is a dimer of acetyl salicylic acid or a pharmaceutically acceptable salt thereof.

3. A method according to claim 1 wherein the dimer of acetyl salicylic acid is 5,5′-methylenebis(2-hydroxybenzoic acid) or 4,4′-diacetoxy-1,1 biphenyl-3,3′ dicarboxylic acid or a pharmaceutically acceptable salt thereof.

4. A method according to claim 1 wherein the complement inhibitor is selected from the group consisting of aurin tricarboxylic acid
(5,5′-((3-carboxyl-4-oxocyclohexa-2,5-d iene)methylene)bis(2-hydroxybenzoic acid)), aurin quadracarboxylic acid
((Z)-3-(3-carboxyl-4-hydroxybenzyl)-5-((3-carboxyl-4-hydroxyphenyl) (3-carboxyl-4-oxocyclohexa-2,5-dien-1-ylidene)methyl)-2-hydroxybenzoic acid), aurin pentacarboxylic acid
((Z)-5-(3-carboxyl-4-hydroxybenzyl)-3-((3-carboxyl-5-((3-carboxyl-4-hydroxyphenyl)(3-carboxyl-4-oxocyclohexa-2,5-dien-1-ylidene)methyl)-2-hydroxybenzyl)-2-hydroxybenzoic acid), aurin hexacarboxylic acid
((Z)-5,5′-((3-carboxyl-5-(3-carboxyl-5-((3-carboxyl-4-hydroxyphenyl)(3-carboxyl-4-oxocyclohexa-2,5-dien-1-ylidene)methyl)-2-hydroxybenzyl)-4-hydroxyphenyl)(hydroxyl) methylene)bis(2-hydroxybenzoic acid)), and pharmaceutically acceptable salts thereof.

5. A method according to claim 1 wherein the complement inhibitor is any combination of aurin tri-, quadra-, penta- and hexacarboxylic acids, or pharmaceutically acceptable salts thereof.

6. A method according to claim 1 wherein the complement inhibitor comprises aurin tri-, quadra-, penta- and hexacarboxylic acids, or pharmaceutically acceptable salts thereof.

7. A method according to claim 1 wherein the complement inhibitor is an inhibitor of formation of the membrane attack complex C5b-9.

8. A method according to claim 1 wherein the complement inhibitor is an ester prodrug of a dimer of acetyl salicylic acid, or aurin tri-, quadra-, penta- or hexacarboxylic acid, or a pharmaceutically acceptable salt thereof.

9. A method according to claim 8 wherein the dimer of acetyl salicylic acid is 5,5′-methylenebis(2-hydroxybenzoic acid) or 4,4′-diacetoxy-1,1 biphenyl-3,3′ dicarboxylic acid.

10. A method according to claim 1 wherein the complement inhibitor is administered orally.

11. A method according to claim 1 wherein the cachexia is cancer cachexia.

12. A method according to claim 1 wherein the cachexia is a non-cancer cachexia associated with a disorder selected from the group consisting of HIV/AIDS, end-stage renal failure, chronic obstructive pulmonary disease (COPD), and geriatric cachexia.

13. A method for diagnosing cachexia in a subject in need thereof, the method comprising:
obtaining a saliva sample from the subject;
stabilizing the saliva sample;
determining a level of TNFα present in the pretreated saliva sample;
comparing the determined value of the TNFα with that of a control value of TNFα derived from a saliva sample of an unaffected control group sample; and
displaying the comparison of the determined value and the control value, wherein the determined value being greater than the control value is indicative of cachexia in the subject.

14. A method according to claim 13 wherein the determined value being at least 1.5 times greater than the control value is indicative of cachexia in the subject.

15. A method according to claim 13 wherein the cachexia is cancer cachexia.

16. A method according to claim 13 wherein the cachexia is a non-cancer cachexia associated with a disorder selected from the group consisting of HIV/AIDS, end-stage renal failure, chronic obstructive pulmonary disease (COPD), and geriatric cachexia.

17. A method according to claim 13 wherein the control value of TNFα ranges from 150 pg/ml to 175 pg/ml.
